(19) United States
(12) Patent Application Publication
(10) Pub. No.: US 2026/0297531 A1
(43) Pub. Date: Oct. 1, 2026

(54) IN VITRO GUT MODEL USING MUCIN-ANALOGS

(71) Applicant: Trustees of Tufts College, MEDFORD, MA (US)

(72) Inventors: David L. Kaplan, Concord, MA (US); Jugal Kishore Sahoo, Belmont, MA (US); Ying Chen, Winchester, MA (US); Sara Rudolph, Cambridge, MA (US)

(21) Appl. No.: 19/400,475

(22) Filed: Nov. 25, 2025

Related U.S. Application Data
(63) Continuation of application No. PCT/US2024/033280, filed on Jun. 10, 2024.
(60) Provisional application No. 63/507,389, filed on Jun. 9, 2023.

(51) Int. Cl. C12N 5/071

(52) U.S. Cl. CPC C12N 5/0679; C12N 2513/00; C12N 2533/50

(57) ABSTRACT

This disclosure relates to an invitro gut model, including an integral layer of intestinal epithelial cells, an artificial mucus atop the integral layer of intestinal epithelial cells, wherein the artificial mucus comprises a silk glycopolymer and optionally a silk polymer lacking sugar substituents, the artificial mucus having a degree of substitution of the silk glycopolymer, a location of substitution of the silk glycopolymer, a concentration of the silk glycopolymer, an overall polymer concentration, an overall silk polymer concentration, an optional concentration of the silk polymer lacking sugar substituents, an optional ratio by weight of the silk glycopolymer and the silk polymer lacking sugar substituents, and/or a silk glycopolymer silk backbone molecular weight is tailored to maintain the integral layer of intestinal epithelial cells for a predetermined length of time, and growth media accessible to the intestinal epithelial cells and tailored to maintain viability of the intestinal epithelial cells.

### CLAIM TO PRIORITY

This application claims benefit of and is a continuation of International Patent Application No. PCT/US 2024/033280 (Attorney Docket No. 2095.0609), filed Jun. 10, 2024, and entitled “IN VITRO GUT MODEL USING MUCIN-ANALOGS,” International Pub. No. WO 2024/254604, which is hereby incorporated by reference in its entirety for all purposes.

International Patent Application No. PCT/US2024/033280 relates to, incorporates by reference for all purposes, and claims priority to U.S. Application Ser. No. 63/507,389 (Attorney Docket No. 2095.0551), filed Jun. 9, 2023.

### BACKGROUND

The major function of gastrointestinal (GI) mucus is protection of epithelial surfaces against stress or damage done by the passage of food and from strong digestion. The GI mucus is responsible for providing lubrication and hydration to facilitate the passage of food and waste through the gastrointestinal tract. Further, mucus serves as a strong barrier for nutrient transport into the epithelium while maintaining protection from potential pathogens, toxins, and harmful byproducts (produced by digestion). Mucus is important for gut and human health as mucus dysregulation can cause many diseases such as ulcerative colitis (UC), Crohn's disease, colorectal cancer, and infections while healthy mucus can maintain gut homeostasis. Epithelial surfaces are vulnerable to many pathogenic strains of microbes with insufficient thickness of mucus and can result in biofouling, microbial invasion, and infection. Due to the enormous utility of mucus in gut homeostasis, human health and different diseases, and proper functioning of the epithelial innate immune system, accurate and efficient models of the gut mucus are needed to accurately assess these functions. Although native mucus can be collected by different methods, they have many limitations related to low yield, heterogeneity in their material properties (e.g., stiffness), and invasiveness of collection.

### SUMMARY

This disclosure relates to an in vitro gut model based on engineered silk glycopolymers and their impact on barrier function, host-bacterial interactions, and drug delivery.

In some aspects, the techniques described herein relate to a cultured composition including: an integral layer of intestinal epithelial cells; an artificial mucus atop the integral layer of intestinal epithelial cells, wherein the artificial mucus includes a silk glycopolymer and optionally a silk polymer lacking sugar substituents, the artificial mucus having a degree of substitution of the silk glycopolymer, a location of substitution of the silk glycopolymer, a concentration of the silk glycopolymer, an overall polymer concentration, an overall silk polymer concentration, an optional concentration of the silk polymer lacking sugar substituents, an optional ratio by weight of the silk glycopolymer and the silk polymer lacking sugar substituents, and/or a silk glycopolymer silk backbone molecular weight is tailored to maintain the integral layer of intestinal epithelial cells for a predetermined length of time; and growth media accessible to the intestinal epithelial cells and tailored to maintain viability of the intestinal epithelial cells.

In aspects, the disclosure herein relates to a method of using the cultured composition disclosed herein, the method including: observing integrity of the integral layer of intestinal epithelial cells.

These and other systems, methods, objects, features, and advantages of the present disclosure will be apparent to those skilled in the art from the following detailed description of the preferred embodiment and the drawings.

All documents mentioned herein are hereby incorporated in their entirety by reference. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context.

### BRIEF DESCRIPTION OF THE FIGURES

The disclosure and the following detailed description of certain embodiments thereof may be understood by reference to the following figures:

FIG. 1: Synthesis of different silk glycopolymers: Different mucin relevant sugars (N-acetyl Galactosamine (GalNAc), N-acetyl Glucosamine (GlcNAc), Sialic acid (NeuNAc), Galactosamine (GalN), Glucosamine (GlcN) are covalently incorporated onto silk chain using a combination of serine carboxylation and carbodiimide coupling chemistry. Carboxylated versions of GalNAc, GlcNAc and NeuNAc are used to facilitate carbodiimide coupling on the already aminated silk (SF(S)-EDA. Aminated versions of Galactose (GalN) and Glucose (GlcN) are used to directly couple to the carboxylated silk (SF(S)-COOH) by carbodiimide coupling in presence of 1-Ethyl-3-[3-dimethylaminopropyl] carbodiimide hydrochloride (EDC) and N-hydroxy succinimide (NHS) at room temperature.

FIG. 2: Schematic of different steps of scaffold preparation for in vitro gut model studies and artificial mucus and microbiome incorporation.

FIG. 3: Left side demonstrate the schematic diagram of transwell set up where SF(S)-GalNAc at different concentration was tested for their trans-epithelial electrical resistance (TEER) resistance and monolayer integrity. a) TEER data for different concentrations of glycopolymers only and in composition with SF after 96 h. TEER data (a) and immunostained monolayer images show SF only (0% SF(S)-GalNAc) and 0.2% of SF(S)-GalNAc maintain the monolayer integrity until 96h.

FIG. 4: Trans-epithelial electrical resistance (TEER) data at different time intervals when 0.2% SF(S)-GalNAc is exposed to commensal E. coli Nissle (EcN) and pathogenic adherent-invasive E. coli (AIEC) cultured in a transwell system with enteroid monolayers.

FIG. 5: a) TEER analysis at different time points for different SF(S)-GalNAc concentrations and composition of SF and SF(S)-GalNAc and control. b and c) Scanning electron microscopy images of the epithelial monolayer when exposed to the control sample (no glycopolymer) and composite mixture of 0.8% SF(S)-GalNAc and 1.2% of SF after seven days. The SEM showed the intact monolayer with microvilli structure (represented with yellow circles and arrows).

FIG. 6: Trans-epithelial electrical resistance (TEER) assessment of different silk glycopolymers alone and in composite settings with SF, SF alone (2%) and control (commercial gastric mucin). The total polymer content for all the solution is at 2 wt% which is like native mucin content in gut mucus.

### DETAILED DESCRIPTION

Before the present disclosure is described in further detail, it is to be understood that the disclosure is not limited to the particular embodiments described. It is also understood that the terminology used herein is for the purpose of describing particular embodiments only, and is not intended to be limiting. The scope of the present disclosure will be limited only by the claims. As used herein, the singular forms “a”, “an”, and “the” include plural embodiments unless the context clearly dictates otherwise.

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” are used as equivalents and may be understood to permit standard variation as would be understood by those of ordinary skill in the art; and (v) where ranges are provided, endpoints are included.

Approximately: as used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Composition: as used herein, may be used to refer to a discrete physical entity that comprises one or more specified components. In general, unless otherwise specified, a composition may be of any form—e.g., gas, gel, liquid, solid, etc. In some embodiments, “composition” may refer to a combination of two or more entities for use in a single embodiment or as part of the same article. It is not required in all embodiments that the combination of entities result in physical admixture, that is, combination as separate co-entities of each of the components of the composition is possible; however many practitioners in the field may find it advantageous to prepare a composition that is an admixture of two or more of the ingredients in a pharmaceutically acceptable carrier, diluent, or excipient, making it possible to administer the component ingredients of the combination at the same time.

Improve, increase, or reduce: as used herein or grammatical equivalents thereof, indicate values that are relative to a baseline measurement, such as a measurement in a similar composition made according to previously known methods.

Substantially: as used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. When two or more ranges for a particular value are recited, this disclosure contemplates all combinations of the upper and lower bounds of those ranges that are not explicitly recited. For example, recitation of a value of between 1 and 10 or between 2 and 9 also contemplates a value of between 1 and 9 or between 2 and 10.

As used herein, “silk fibroin” refers to silk fibroin protein whether produced by silkworm, spider, or other insect, or otherwise generated (Lucas et al., Adv. Protein Chem., 13:107-242 (1958)). Any type of silk fibroin can be used in different embodiments described herein. Silk fibroin produced by silkworms, such as Bombyx mori, is the most common and represents an earth-friendly, renewable resource. For instance, silk fibroin used in a silk film may be attained by extracting sericin from the cocoons of B. mori. Organic silkworm cocoons are also commercially available. There are many different silks, however, including spider silk (e.g., obtained from Nephila clavipes), transgenic silks, genetically engineered silks, such as silks from bacteria, yeast, mammalian cells, transgenic animals, or transgenic plants, and variants thereof, that can be used. See, e.g., WO 97/08315 and U.S. Pat. No. 5,245,012, each of which is incorporated herein by reference in their entireties.

This disclosure relates to the gut microbiome and investigating the potential impact of mucin analogs on barrier protection (presence and absence of microbes) and deciphering the data to understand gut related disease pathology (e.g., Inflammatory Bowel Diseases. Synthetic analogs of gut mucus, prepared in a laboratory set up, may find tremendous utility to design in vitro gut models and decipher their structure-properties-function related to host-microbe interactions and different disease pathology related to the gut.

Disclosed here is an in vitro gut model with engineered mucus barrier based on silk glycopolymers for studying host-bacterial interactions and drug delivery. Also disclosed herein is an assessment of the ability of various mucin-inspired silk glycopolymers to barrier resistance and monolayer integrity and a comparison of their performance with native gut mucus. The disclosure herein encompasses an evaluation of the cytotoxicity of glycopolymers using Caco-2 cells and human intestinal organoids, followed by examining whether silk glycopolymers can effectively emulate the selective barrier properties of native mucus by serving as a physical separator between the intestinal epithelium and microbiome, while still facilitating the passage of beneficial substances to enable crucial host-microbial interactions. Different glycopolymer variants were screened based on silk protein and their potential to maintain monolayer integrity of epithelial surface was assessed.

The disclosure herein relates to a cultured composition. In some examples, the cultured composition includes an integral layer of intestinal epithelial cells and an artificial mucus atop the integral layer of intestinal epithelial cells. The artificial mucus comprises a silk glycopolymer and optionally a silk polymer lacking sugar substituents. Aspects of the artificial mucus may be tailored to maintain the integral layer of intestinal epithelial cells for a predetermined length of time, including a degree of substitution of the silk glycopolymer, a location of substitution of the silk glycopolymer, a concentration of the silk glycopolymer, an overall polymer concentration, an overall silk polymer concentration, an optional concentration of the silk polymer lacking sugar substituents, an optional ratio by weight of the silk glycopolymer and the silk polymer lacking sugar substituents, and/or a silk glycopolymer silk backbone molecular weight. The cultured composition also includes a growth media accessible to the intestinal epithelial cells. The growth media is tailored to maintain viability of the intestinal epithelial cells.

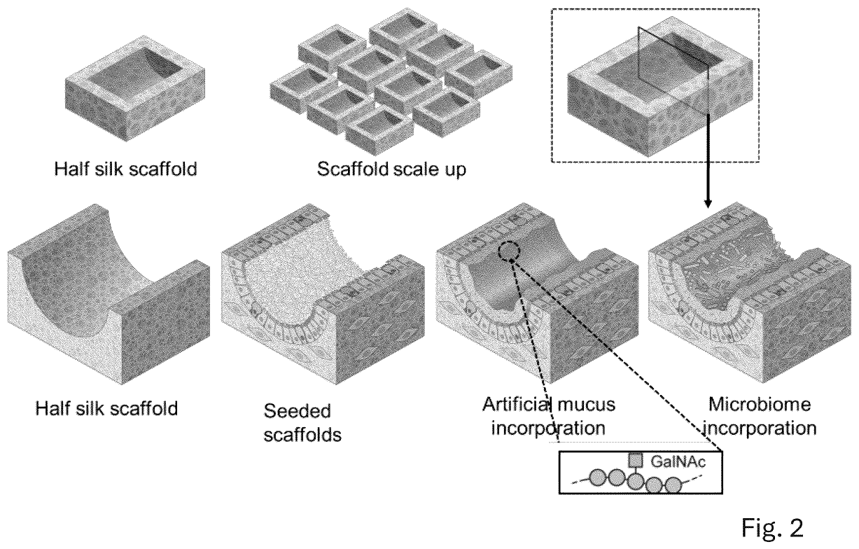

In some examples, the culture composition further includes a three-dimensional tissue scaffold (FIG. 2), wherein the integral layer of intestinal epithelial cells is affixed to the three-dimensional tissue scaffold. The three-dimensional tissue scaffold may be a tubular scaffold, such as a partial tubular structure, a half tubular structure, or a full tubular structure. The three-dimensional tissue scaffold may be a silk fibroin tissue scaffold.

In some examples, the cultured composition further includes a plurality of intestinal organoids physiologically associated with the integral layer of intestinal epithelial cells. In an example, the plurality of intestinal organoids is patient-specific. The intestinal organoids of the plurality of intestinal organoids are harvested from a specific patient or grown from cells from the specific patient. The specific patient has a predefined disease state, and the predefined disease state is inflammatory bowel disease (IBD).

In some examples, the integral layer of intestinal epithelial cells is an integral layer of Caco-2 cells.

In some examples of the cultured composition, the silk glycopolymer has a degree of sugar substitution of between 0.5% and 40%. In some examples of the cultured composition, the silk glycopolymer has a degree of sugar substitution of at least 0.5%. In some examples of the cultured composition, the silk glycopolymer has a degree of sugar substitution of at least 1.0%. In some examples of the cultured composition, the silk glycopolymer has a degree of sugar substitution of at least 1.5%. In some examples of the cultured composition, the silk glycopolymer has a degree of sugar substitution of at least 2.0%. In some examples of the cultured composition, the silk glycopolymer has a degree of sugar substitution of at least 2.5%. In some examples of the cultured composition, the silk glycopolymer has a degree of sugar substitution of at least 3.0%. In some examples of the cultured composition, the silk glycopolymer has a degree of sugar substitution of at least 3.5%. In some examples of the cultured composition, the silk glycopolymer has a degree of sugar substitution of at least 4.0%. In some examples of the cultured composition, the silk glycopolymer has a degree of sugar substitution of at least 4.5%. In some examples of the cultured composition, the silk glycopolymer has a degree of sugar substitution of at least 5.0%. In some examples of the cultured composition, the silk glycopolymer has a degree of sugar substitution of at least 7.5%. In some examples of the cultured composition, the silk glycopolymer has a degree of sugar substitution of at least 10.0%. In some examples of the cultured composition, the silk glycopolymer has a degree of sugar substitution of or at least 15.0%. In some examples of the cultured composition, the silk glycopolymer has a degree of sugar substitution of at most 40.0%. In some examples of the cultured composition, the silk glycopolymer has a degree of sugar substitution of at most 35.0%. In some examples of the cultured composition, the silk glycopolymer has a degree of sugar substitution of at most 30.0%. In some examples of the cultured composition, the silk glycopolymer has a degree of sugar substitution of at most 25.0%. In some examples of the cultured composition, the silk glycopolymer has a degree of sugar substitution of at most 20.0%. In some examples of the cultured composition, the silk glycopolymer has a degree of sugar substitution of at most 15.0%. In some examples of the cultured composition, the silk glycopolymer has a degree of sugar substitution of at most 10.0%. In some examples of the cultured composition, the silk glycopolymer has a degree of sugar substitution of at most 5.0%.

In an example, the silk glycopolymer has a degree of sugar substitution of between 0.5% and 5.0%. In an example, the silk glycopolymer has a degree of sugar substitution of between 10.0% and 40.0%. In an example, the silk glycopolymer has a degree of sugar substitution of between 5.0% and 20.0%.

In some examples of the cultured composition, the silk glycopolymer is an O-glycan-substituted silk fibroin.

In some examples of the cultured composition, the silk glycopolymer includes a GalNAc-modified silk fibroin.

In some examples of the cultured composition, the silk glycopolymer includes a GlcN-modified silk fibroin.

In some examples of the cultured composition, the silk glycopolymer includes a GalN-modified silk fibroin.

In some examples of the cultured composition, the silk glycopolymer includes SF(D, E)-GalNAc.

In some examples of the cultured composition, the silk glycopolymer includes SF(S)-GalN.

In some examples of the cultured composition, the silk glycopolymer includes SF(S)-GlcN.

In the example where the silk glycopolymer includes SF(S)-GlcN, the artificial mucus has a concentration of the silk polymer lacking sugar substituents in a weight percentage of between 0.1% and 4.0%, including but not limited to, at least 0.1%, at least 0.3%, at least 0.5%, at least 1.0% or at least 1.5% and at most 4.0%, at most 3.0%, at most 2.5%, at most 2.0%, or at most 1.5%. In some cases, the artificial mucus has a concentration of the silk polymer lacking sugar substituents in a weight percentage of at least 0.1%. In some cases, the artificial mucus has a concentration of the silk polymer lacking sugar substituents in a weight percentage of at least 0.3%. In some cases, the artificial mucus has a concentration of the silk polymer lacking sugar substituents in a weight percentage of at least 0.5%. In some cases, the artificial mucus has a concentration of the silk polymer lacking sugar substituents in a weight percentage of at least 1.0%. In some cases, the artificial mucus has a concentration of the silk polymer lacking sugar substituents in a weight percentage of at least 1.5%. In some cases, the artificial mucus has a concentration of the silk polymer lacking sugar substituents in a weight percentage of at most 4.0%. In some cases, the artificial mucus has a concentration of the silk polymer lacking sugar substituents in a weight percentage of at most 3.0%. In some cases, the artificial mucus has a concentration of the silk polymer lacking sugar substituents in a weight percentage of at most 2.5%. In some cases, the artificial mucus has a concentration of the silk polymer lacking sugar substituents in a weight percentage of at most 2.0%. In some cases, the artificial mucus has a concentration of the silk polymer lacking sugar substituents in a weight percentage of at most 1.5%.

In the example where the silk glycopolymer includes SF(S)-GlcN, a ratio by weight of the silk glycopolymer to the silk polymer lacking sugar substituents is between 1:5 and 5:1, including but not limited to, at least 1:5, at least 1:4, at least 1:3, at least 1:2, at least 1:1, at least 2:1, at least 3:1, or at least 4:1 and at most 5:1, at most 4:1, at most 3:1, at most 2:1, at most 1:1, at most 1:2, at most 1:3, or at most 1:4. In some cases, the ratio by weight of the silk glycopolymer to the silk polymer lacking sugar substituents is at least 1:5. In some cases, the ratio by weight of the silk glycopolymer to the silk polymer lacking sugar substituents is at least 1:4. In some cases, the ratio by weight of the silk glycopolymer to the silk polymer lacking sugar substituents is at least 1:3. In some cases, the ratio by weight of the silk glycopolymer to the silk polymer lacking sugar substituents is at least 1:2. In some cases, the ratio by weight of the silk glycopolymer to the silk polymer lacking sugar substituents is at least 1:1. In some cases, the ratio by weight of the silk glycopolymer to the silk polymer lacking sugar substituents is at least 2:1. In some cases, the ratio by weight of the silk glycopolymer to the silk polymer lacking sugar substituents is at least 3:1. In some cases, the ratio by weight of the silk glycopolymer to the silk polymer lacking sugar substituents is at least 4:1. In some cases, the ratio by weight of the silk glycopolymer to the silk polymer lacking sugar substituents is at most 5:1. In some cases, the ratio by weight of the silk glycopolymer to the silk polymer lacking sugar substituents is at most 4:1. In some cases, the ratio by weight of the silk glycopolymer to the silk polymer lacking sugar substituents is at most 3:1. In some cases, the ratio by weight of the silk glycopolymer to the silk polymer lacking sugar substituents is at most 2:1. In some cases, the ratio by weight of the silk glycopolymer to the silk polymer lacking sugar substituents is at most 1:1. In some cases, the ratio by weight of the silk glycopolymer to the silk polymer lacking sugar substituents is at most 1:2. In some cases, the ratio by weight of the silk glycopolymer to the silk polymer lacking sugar substituents is at most 1:3. In some cases, the ratio by weight of the silk glycopolymer to the silk polymer lacking sugar substituents is at most 1:4.

In examples where the silk glycopolymer includes SF(S)-GlcN, the silk polymer lacking sugar substituents can be unmodified silk fibroin.

In some examples of the cultured composition, the artificial mucus has a total polymer concentration in a weight percentage of between 0.5% and 10.0%, including but not limited to, at least 0.5%, at least 1.0%, at least 1.5%, or at least 2.0% and at most 10.0%, at most 7.5%, at most 6.0%, at most 5.0%, or at most 4.0%. In some examples, the artificial mucus has a total polymer concentration in a weight percentage of between 0.5% and 5.0%. In some examples, the artificial mucus has a total polymer concentration in a weight percentage of between 6.0% and 10.0%. In some examples, the artificial mucus has a total polymer concentration in a weight percentage of between 2.0% and 7.5%. In some cases, the artificial mucus has a total polymer concentration in a weight percentage of at least 0.5%. In some cases, the artificial mucus has a total polymer concentration in a weight percentage of at least 1.0%. In some cases, the artificial mucus has a total polymer concentration in a weight percentage of at least 1.5%. In some cases, the artificial mucus has a total polymer concentration in a weight percentage of or at least 2.0%. In some cases, the artificial mucus has a total polymer concentration in a weight percentage of at most 10.0%. In some cases, the artificial mucus has a total polymer concentration in a weight percentage of at most 7.5%. In some cases, the artificial mucus has a total polymer concentration in a weight percentage of at most 6.0%. In some cases, the artificial mucus has a total polymer concentration in a weight percentage of at most 5.0%. In some cases, the artificial mucus has a total polymer concentration in a weight percentage of at most 4.0%.

In some examples of the cultured composition, the artificial mucus has an overall silk polymer concentration in a weight percentage of between 0.5% and 10.0%, including but not limited to, at least 0.5%, at least 1.0%, at least 1.5%, or at least 2.0% and at most 10.0%, at most 7.5%, at most 6.0%, at most 5.0%, or at most 4.0%. In some examples, the artificial mucus has an overall silk polymer concentration in a weight percentage of between 0.5% and 5.0%. In some examples, the artificial mucus has an overall silk polymer concentration in a weight percentage of between 6.0% and 10.0%. In some examples, the artificial mucus has an overall silk polymer concentration in a weight percentage of between 2.0% and 7.5%. The artificial mucus has an overall silk polymer concentration in a weight percentage of at least 0.5%. The artificial mucus has an overall silk polymer concentration in a weight percentage of at least 1.0%. The artificial mucus has an overall silk polymer concentration in a weight percentage of at least 1.5%. The artificial mucus has an overall silk polymer concentration in a weight percentage of at least 2.0%. The artificial mucus has an overall silk polymer concentration in a weight percentage of at most 10.0%. The artificial mucus has an overall silk polymer concentration in a weight percentage of at most 7.5%. The artificial mucus has an overall silk polymer concentration in a weight percentage of at most 6.0%. The artificial mucus has an overall silk polymer concentration in a weight percentage of at most 5.0%. The artificial mucus has an overall silk polymer concentration in a weight percentage of at most 4.0%.

In some examples of the cultured composition, the artificial mucus has a concentration of the silk glycopolymer in a weight percentage of between 0.5% and 10.0%, including but not limited to, at least 0.5%, at least 1.0%, at least 1.5%, or at least 2.0% and at most 10.0%, at most 7.5%, at most 6.0%, at most 5.0%, or at most 4.0%. In some examples, the artificial mucus has a concentration of the silk glycopolymer in a weight percentage of between 0.5% and 5.0%. In some examples, the artificial mucus has a concentration of the silk glycopolymer in a weight percentage of between 6.0% and 10.0%. In some examples, the artificial mucus has a concentration of the silk glycopolymer in a weight percentage of between 2.0% and 7.5%. The artificial mucus has a concentration of the silk glycopolymer in a weight percentage of at least 0.5%. The artificial mucus has a concentration of the silk glycopolymer in a weight percentage of at least 1.0%. The artificial mucus has a concentration of the silk glycopolymer in a weight percentage of at least 1.5%. The artificial mucus has a concentration of the silk glycopolymer in a weight percentage of at least 2.0%. The artificial mucus has a concentration of the silk glycopolymer in a weight percentage of at most 10.0%. The artificial mucus has a concentration of the silk glycopolymer in a weight percentage of at most 7.5%. The artificial mucus has a concentration of the silk glycopolymer in a weight percentage of at most 6.0%. The artificial mucus has a concentration of the silk glycopolymer in a weight percentage of at most 5.0%. The artificial mucus has a concentration of the silk glycopolymer in a weight percentage of at most 4.0%.

In some examples of the cultured composition, the artificial mucus comprises the silk polymer lacking sugar substituents. In examples, the silk polymer lacking sugar substituents is unmodified silk fibroin.

In examples of the cultured composition, the artificial mucus is free of organic solvents.

In examples of the cultured composition, the cultured composition is free of organic solvents.

In examples of the cultured composition, the artificial mucus has one or more mechanical properties within 5% of a respective native mucus mechanical property.

In examples of the cultured composition, the integral layer of intestinal epithelial cells maintains integrity for at least 7 days.

In examples of the cultured composition, the artificial mucus is tailored to maintain integrity of the integral layer of intestinal epithelial cells in a microbial environment that is otherwise destructive to the integral layer of intestinal epithelial cells.

Disclosed herein is a method of using the cultured composition disclosed herein, the method including observing integrity of the integral layer of intestinal epithelial cells. The method may further include assessing performance of the artificial mucus based on the observed integrity. The observing and/or assessing can include measuring a transepithelial electrical property of the integral layer of intestinal epithelial cells. The observing and/or assessing can include visual inspection and/or imaging of the integral layer of intestinal epithelial cells.

According to various embodiments, a variety of functionalizing agents may be used with the silk-containing embodiments described herein (e.g., silk membrane, silk composition, silk matrix, silk foam, silk microsphere, etc.). It should be understood that the examples herein may recite one or a few silk-containing embodiments but are applicable to any silk-containing embodiment, as applicable. In some embodiments, a functionalizing agent may be any compound or molecule that facilitates the attachment to and/or development (e.g., growth) of one or more endothelial cells on a silk membrane. In some embodiments, a functionalizing agent may be any compound or molecule that facilitates the attachment and/or development (e.g., growth) of one or more megakaryocytes and/or hematopoietic progenitor cells on a silk matrix and/or silk membrane. In some embodiments, a functionalizing agent may be or comprise an agent suitable for facilitating the production of one or more of white blood cells and red blood cells.

In some embodiments, a functionalizing agent may be or comprise a cell attachment mediator and/or an extracellular matrix protein, for example: collagen (e.g., collagen type I, collagen type III, collagen type IV or collagen type VI), elastin, fibronectin, vitronectin, laminin, fibrinogen, von Willebrand factor, proteoglycans, decorin, perlecan, nidogen, hyaluronan, and/or peptides containing known integrin binding domains e.g. “RGD” integrin binding sequence, or variations thereof, that are known to affect cellular attachment.

In some embodiments, a functionalizing agent may be any soluble molecule produced by endothelial cells. Non-limiting examples include fibroblast growth factor-1 (FGF1) and vascular endothelial growth factors (VEGF).

According to some embodiments, a plurality of functionalizing agents may be used. For example, in some embodiments wherein production of platelets is desired, provided compositions may comprise the use of laminin, fibronectin and/or fibrinogen, and type IV collagen in order to facilitate the attachment and growth of endothelial cells on a silk membrane (e.g., a porous silk membrane) and/or attachment of megakaryocytes to a silk matrix.

In some embodiments, a functionalizing agent may be embedded or otherwise associated with a silk membrane and/or silk matrix such that at least a portion of the functionalizing agent is surrounded by a silk membrane and/or silk matrix as contrasted to a functionalizing agent simply being positioned along the surface of a silk membrane and/or silk matrix. In some embodiments, a functionalizing agent is distributed along and/or incorporated in substantially the entire surface area of a silk membrane/silk wall. In some embodiments, a functionalizing agent is distributed and/or incorporated only at one or more discrete portions of a silk membrane/wall and/or silk matrix. In some embodiments, a functionalizing agent is distributed in and/or along at least one of the lumen-facing side of a silk wall and the matrix-facing side of a silk wall.

According to various embodiments, any application-appropriate amount of one or more functionalizing agents may be used. In some embodiments, the amount of an individual functionalizing agent may be between about 1 μg/ml and 1,000 μg/ml (e.g., between about 2 and 1,000, 5 and 1,000, 10 and 1,000, 10 and 500, 10 and 100 μg/ml). In some embodiments, the amount of an individual functionalizing agent may be at least 1 μg/ml (e.g., at least 5, 10, 15, 20 25, 50, 100, 200, 300 400, 500, 600, 700, 800, or 900 μg/ml). In some embodiments, the amount of an individual functionalizing agent is at most 1,000 μg/ml (e.g., 900, 800, 700, 600, 500, 400, 300 200, 100, 90, 80, 70, 60, 50, 40, 30, 20, 10, or 5 μg/ml).

In some aspects, the composition comprises one or more sensing agents, such as a sensing dye. The sensing agents/sensing dyes are environmentally sensitive and produce a measurable response to one or more environmental factors. In some aspects, the environmentally-sensitive agent or dye may be present in the composition in an effective amount to alter the composition from a first chemical-physical state to a second chemical-physical state in response to an environmental parameter (e.g., a change in pH, light intensity or exposure, temperature, pressure or strain, voltage, physiological parameter of a subject, and/or concentration of chemical species in the surrounding environment) or an externally applied stimulus (e.g., optical interrogation, acoustic interrogation, and/or applied heat). In some cases, the sensing dye is present to provide one optical appearance under one given set of environmental conditions and a second, different optical appearance under a different given set of environmental conditions. Suitable concentrations for the sensing agents described herein can be the concentrations for the colorants and additives described elsewhere herein. A person having ordinary skill in the chemical sensing arts can determine a concentration that is appropriate for use in a sensing application of the inks described herein.

In some aspects, the first and second chemical-physical state may be a physical property of the composition, such as mechanical property, a chemical property, an acoustical property, an electrical property, a magnetic property, an optical property, a thermal property, a radiological property, or an organoleptic property. Exemplary sensing dyes or agents include, but are not limited to, a pH sensitive agent, a thermal sensitive agent, a pressure or strain sensitive agent, a light sensitive agent, or a potentiometric agent.

Exemplary pH sensitive dyes or agents include, but are not limited to, cresol red, methyl violet, crystal violet, ethyl violet, malachite green, methyl green, 2-(p-dimethylaminophenylazo) pyridine, paramethyl red, metanil yellow, 4-phenylazodiphenylamine, thymol blue, metacresol purple, orange IV, 4-o-Tolylazo-o-toluindine, quinaldine red, 2,4-dinitrophenol, erythrosine disodium salt, benzopurpurine 4B, N,N-dimethyl-p-(m-tolylazo) aniline, p-dimethylaminoazobenene, 4,4′-bis(2-amino-1-naphthylazo)-2,2′-stilbenedisulfonic acid, tetrabromophenolphthalein ethyl ester, bromophenol blue, Congo red, methyl orange, ethyl orange, 4-(4-dimethylamino-1-naphylazo)-3-methoxybenesulfonic acid, bromocresol green, resazurin, 4-phenylazo-1-napthylamine, ethyl red 2-([-dimethylaminophenyazo) pyridine, 4-(p-ethoxypehnylazo)-m-phenylene-diamine monohydrochloride, resorcin blue, alizarin red S, methyl red, propyl red, bromocresol purple, chlorophenol red, p-nitrophenol, alizarin 2-(2,4-dinitrophenylazo) 1-napthol-3,6-disulfonic acid, bromothymol blue, 6,8-dinitro-2,4-(1H) quinazolinedione, brilliant yellow, phenol red, neutral red, m-nitrophenol, cresol red, turmeric, metacresol purple, 4,4′-bis(3-amino-1-naphthylazo)-2,2′-stilbenedisulfonic acid, thymol blue, p-naphtholbenzein, phenolphthalein, o-cresolphthalein, ethyl bis(2,4-dimethylphenyl) ethanoate, thymolphthalein, nitrazine yellow, alizarin yellow R, alizarin, p-(2,4-dihydroxyphenylazo) benzenesulfonic acid, 5,5′-indigodisulfonic acid, 2,4,6-trinitrotoluene, 1,3,5-trinitrobenezne, and clayton yellow.

Exemplary light responsive dyes or agents include, but are not limited to, photochromic compounds or agents, such as triarylmethanes, stilbenes, azasilbenes, nitrones, fulgides, spiropyrans, napthopyrans, spiro-oxzines, quinones, derivatives and combinations thereof.

Exemplary potentiometric dyes include, but are not limited to, substituted amiononaphthylehenylpridinium (ANEP) dyes, such as di-4-ANEPPS, di-8-ANEPPS, and N-(4-Sulfobutyl)-4-(6-(4-(Dibutylamino)phenyl)hexatrienyl)Pyridinium (RH237).

Exemplary temperature sensitive dyes or agents include, but are not limited to, thermochromic compounds or agents, such as thermochromic liquid crystals, leuco dyes, fluoran dyes, octadecylphosphonic acid.

Exemplary pressure or strain sensitive dyes or agents include, but are not limited to, spiropyran compounds and agents.

Exemplary chemi-sensitive dyes or agents include, but are not limited to, antibodies such as immunoglobulin G (IgG) which may change color from blue to red in response to bacterial contamination.

In some aspects, the compositions comprise one or more additive, dopant, or biologically active agent suitable for a desired intended purpose. In some aspects, the additive or dopant may be present in the composition in an amount effective to impart an optical or organoleptic property to the composition. Exemplary additives or dopants that impart optical or organoleptic properties include, but are not limited to, dyes/pigments, flavorants, aroma compounds, granular or fibrous fillers.

Additionally or alternatively, the additive, dopant, or biologically active agent may be present in the composition in an amount effective to “functionalize” the composition to impart a desired mechanical property or added functionality to the composition. Exemplary additive, dopants, or biologically active agent that impart the desired mechanical property or added functionality include, but are not limited to: environmentally sensitive/sensing dyes; active biomolecules; conductive or metallic particles; micro and nanofibers (e.g., silk nanofibers for reinforcement, carbon nanofibers); nanotubes; inorganic particles (e.g., hydroxyapatite, tricalcium phosphate, bioglasses); drugs (e.g., antibiotics, small molecules or low molecular weight organic compounds); proteins and fragments or complexes thereof (e.g., enzymes, antigens, antibodies and antigen-binding fragments thereof); DNA/RNA (e.g., siRNA, miRNA, mRNA); cells and fractions thereof (viruses and viral particles; prokaryotic cells such as bacteria; eukaryotic cells such as mammalian cells and plant cells; fungi).

In some aspects, the additive or dopant comprises a conductive additive. Exemplary conductive additives include, but are not limited to graphite, graphite powder, carbon nanotubes, and metallic particles or nanoparticles, such as gold nanoparticles. In some aspects, the conductive additive is biocompatible and non-toxic.

In some aspects, the additive is a biologically active agent. The term “biologically active agent” as used herein refers to any molecule which exerts at least one biological effect in vivo. For example, the biologically active agent can be a therapeutic agent to treat or prevent a disease state or condition in a subject. Biologically active agents include, without limitation, organic molecules, inorganic materials, proteins, peptides, nucleic acids (e.g., genes, gene fragments, gene regulatory sequences, and antisense molecules), nucleoproteins, polysaccharides, glycoproteins, and lipoproteins. Classes of biologically active compounds that can be incorporated into the composition provided herein include, without limitation, anticancer agents, antibiotics, analgesics, anti-inflammatory agents, immunosuppressants, enzyme inhibitors, antihistamines, anti-convulsants, hormones, muscle relaxants, antispasmodics, ophthalmic agents, prostaglandins, anti-depressants, anti-psychotic substances, trophic factors, osteoinductive proteins, growth factors, and vaccines.

The term “active agent” may also be used herein to refer to a biological sample (e.g., a sample of tissue or fluid, such as for instance blood) or a component thereof, and/or to a biologically active entity or compound, and/or to a structurally or functionally labile entity.

Exemplary active agents include, but are not limited to, therapeutic agents, diagnostic agents (e.g., contrast agents), and any combinations thereof. In some embodiments, the active agent present in a silk matrix (e.g., a silk microsphere), composition, or the like can include a labile active agent, e.g., an agent that can undergo chemical, physical, or biological change, degradation and/or deactivation after exposure to a specified condition, e.g., high temperatures, high humidity, light exposure, and any combinations thereof. In some embodiments, the active agent present in the silk matrix (e.g., a silk microsphere), composition, or the like can include a temperature-sensitive active agent, e.g., an active agent that will lose at least about 30% or more, of its original activity or bioactivity, upon exposure to a temperature of at least about 10° C. or above, including at least about 15° C. or above, at least about room temperature or above, or at least about body temperature (e.g., about 37° C.) or above.

The active agent can be generally present in the silk matrix (e.g., a silk microsphere), composition, or the like in an amount of about 0.01% (w/w) to about 70% (w/w), or about 0.1% (w/w) to about 50% (w/w), or about 1% (w/w) to about 30% (w/w). The active agent can be present on a surface of the silk matrix (e.g., a silk microsphere), composition, or the like and/or encapsulated and dispersed in the silk matrix (e.g., a silk microsphere), composition, or the like homogeneously or heterogeneously or in a gradient. In some embodiments, the active agent can be added into the silk solution, which is then subjected to the methods described herein for preparing a silk matrix (e.g., a silk microsphere), composition, or the like. In some embodiments, the active agent can be coated on a surface of the silk matrix (e.g., a silk microsphere), composition, or the like. In some embodiments, the active agent can be loaded in a silk matrix (e.g., a silk microsphere), composition, or the like by incubating the silk microsphere in a solution of the active agent for a period of time, during which an amount of the active agent can diffuse into the silk matrix (e.g., a silk microsphere), composition, or the like, and thus distribute within the silk matrix (e.g., a silk microsphere), composition, or the like.

In some aspects, the additive is a therapeutic agent. As used herein, the term “therapeutic agent” means a molecule, group of molecules, complex or substance administered to an organism for diagnostic, therapeutic, preventative medical, or veterinary purposes. As used herein, the term “therapeutic agent” includes a “drug” or a “vaccine.” This term include externally and internally administered topical, localized and systemic human and animal pharmaceuticals, treatments, remedies, nutraceuticals, cosmeceuticals, biologicals, devices, diagnostics and contraceptives, including preparations useful in clinical and veterinary screening, prevention, prophylaxis, healing, wellness, detection, imaging, diagnosis, therapy, surgery, monitoring, cosmetics, prosthetics, forensics and the like. This term can also be used in reference to agriceutical, workplace, military, industrial and environmental therapeutics or remedies comprising selected molecules or selected nucleic acid sequences capable of recognizing cellular receptors, membrane receptors, hormone receptors, therapeutic receptors, microbes, viruses or selected targets comprising or capable of contacting plants, animals and/or humans. This term can also specifically include nucleic acids and compounds comprising nucleic acids that produce a therapeutic effect, for example deoxyribonucleic acid (DNA), ribonucleic acid (RNA), nucleic acid analogues (e.g., locked nucleic acid (LNA), peptide nucleic acid (PNA), xeno nucleic acid (XNA)), or mixtures or combinations thereof, including, for example, DNA nanoplexes, siRNA, microRNA, shRNA, aptamers, ribozymes, decoy nucleic acids, antisense nucleic acids, RNA activators, and the like. Generally, any therapeutic agent can be included in the composition provided herein.

The term “therapeutic agent” also includes an agent that is capable of providing a local or systemic biological, physiological, or therapeutic effect in the biological system to which it is applied. For example, the therapeutic agent can act to control infection or inflammation, enhance cell growth and tissue regeneration, control tumor growth, act as an analgesic, promote anti-cell attachment, and enhance bone growth, among other functions. Other suitable therapeutic agents can include anti-viral agents, hormones, antibodies, or therapeutic proteins. Other therapeutic agents include prodrugs, which are agents that are not biologically active when administered but, upon administration to a subject are converted to biologically active agents through metabolism or some other mechanism. Additionally, a silk-based drug delivery composition can contain one therapeutic agent or combinations of two or more therapeutic agents.

A therapeutic agent can include a wide variety of different compounds, including chemical compounds and mixtures of chemical compounds, e.g., small organic or inorganic molecules; saccharines; oligosaccharides; polysaccharides; biological macromolecules, e.g., peptides, proteins, and peptide analogs and derivatives; peptidomimetics; antibodies and antigen binding fragments thereof; nucleic acids; nucleic acid analogs and derivatives; an extract made from biological materials such as bacteria, plants, fungi, or animal cells; animal tissues; naturally occurring or synthetic compositions; and any combinations thereof. In some aspects, the therapeutic agent is a small molecule.

The term “bioactivity,” as used herein in reference to an active agent, generally refers to the ability of an active agent to interact with a biological target and/or to produce an effect on a biological target. For example, bioactivity can include, without limitation, elicitation of a stimulatory, inhibitory, regulatory, toxic or lethal response in a biological target. The biological target can be a molecule or a cell. For example, a bioactivity can refer to the ability of an active agent to modulate the effect/activity of an enzyme, block a receptor, stimulate a receptor, modulate the expression level of one or more genes, modulate cell proliferation, modulate cell division, modulate cell morphology, or any combination thereof. In some instances, a bioactivity can refer to the ability of a compound to produce a toxic effect in a cell. Exemplary cellular responses include, but are not limited to, lysis, apoptosis, growth inhibition, and growth promotion; production, secretion, and surface expression of a protein or other molecule of interest by the cell; membrane surface molecule activation including receptor activation; transmembrane ion transports; transcriptional regulations; changes in viability of the cell; changes in cell morphology; changes in presence or expression of an intracellular component of the cell; changes in gene expression or transcripts; changes in the activity of an enzyme produced within the cell; and changes in the presence or expression of a ligand and/or receptor (e.g., protein expression and/or binding activity). Methods for assaying different cellular responses are well known to one of skill in the art, e.g., western blot for determining changes in presence or expression of an endogenous protein of the cell, or microscopy for monitoring the cell morphology in response to the active agent, or FISH and/or qPCR for the detection and quantification of changes in nucleic acids. Bioactivity can be determined in some embodiments, for example, by assaying a cellular response.

In reference to an antibody, the term “bioactivity” includes, but is not limited to, epitope or antigen binding affinity, the in vivo and/or in vitro stability of the antibody, the immunogenic properties of the antibody, e.g., when administered to a human subject, and/or the ability to neutralize or antagonize the bioactivity of a target molecule in vivo or in vitro. The aforementioned properties or characteristics can be observed or measured using art-recognized techniques including, but not limited to, scintillation proximity assays, ELISA, ORIGEN immunoassay (IGEN), fluorescence quenching, fluorescence ELISA, competitive ELISA, SPR analysis including, but not limited to, SPR analysis using a BIAcore biosenser, in vitro and in vivo neutralization assays (see, for example, International Publication No. WO 2006/062685), receptor binding, and immunohistochemistry with tissue sections from different sources including human, primate, or any other source as needed. In reference to an immunogen, the “bioactivity” includes immunogenicity, the definition of which is discussed in detail later. In reference to a virus, the “bioactivity” includes infectivity, the definition of which is discussed in detail later. In reference to a contrast agent, e.g., a dye, the “bioactivity” refers to the ability of a contrast agent when administered to a subject to enhance the contrast of structures or fluids within the subject's body. The bioactivity of a contrast agent also includes, but is not limited to, its ability to interact with a biological environment and/or influence the response of another molecule under certain conditions.

As used herein, the term “small molecule” can refer to compounds that are “natural product-like,” however, the term “small molecule” is not limited to “natural product-like” compounds. Rather, a small molecule is typically characterized in that it contains several carbon-carbon bonds, and has a molecular weight of less than 5000 Daltons (5 kDa), preferably less than 3 kDa, still more preferably less than 2 kDa, and most preferably less than 1 kDa. In some cases it is preferred that a small molecule have a molecular weight equal to or less than 700 Daltons.

Exemplary therapeutic agents include, but are not limited to, those found in Harrison's Principles of Internal Medicine, 13th Edition, Eds. T. R. Harrison et al. McGraw-Hill N.Y., NY; Physicians' Desk Reference, 50th Edition, 1997, Oradell New Jersey, Medical Economics Co.; Pharmacological Basis of Therapeutics, 8th Edition, Goodman and Gilman, 1990; United States Pharmacopeia, The National Formulary, ETSP XII NF XVII, 1990, the complete contents of all of which are incorporated herein by reference.

Therapeutic agents include the herein disclosed categories and specific examples. It is not intended that the category be limited by the specific examples. Those of ordinary skill in the art will recognize also numerous other compounds that fall within the categories and that are useful according to the present disclosure. Examples include a radiosensitizer, a steroid, a xanthine, a beta-2-agonist bronchodilator, an anti-inflammatory agent, an analgesic agent, a calcium antagonist, an angiotensin-converting enzyme inhibitors, a beta-blocker, a centrally active alpha-agonist, an alpha-1-antagonist, an anticholinergic/antispasmodic agent, a vasopressin analogue, an anti arrhythmic agent, an antiparkinsonian agent, an antiangina/antihypertensive agent, an anticoagulant agent, an antiplatelet agent, a sedative, an ansiolytic agent, a peptidic agent, a biopolymeric agent, an antineoplastic agent, a laxative, an antidiarrheal agent, an antimicrobial agent, an antifungal agent, a vaccine, a protein, or a nucleic acid. In a further aspect, the pharmaceutically active agent can be coumarin, albumin, steroids such as betamethasone, dexamethasone, methylprednisolone, prednisolone, prednisone, triamcinolone, budesonide, hydrocortisone, and pharmaceutically acceptable hydrocortisone derivatives; xanthines such as theophylline and doxophylline; beta-2-agonist bronchodilators such as salbutamol, fenterol, clenbuterol, bambuterol, salmeterol, fenoterol; antiinflammatory agents, including antiasthmatic anti-inflammatory agents, antiarthritis antiinflammatory agents, and non-steroidal antiinflammatory agents, examples of which include but are not limited to sulfides, mesalamine, budesonide, salazopyrin, diclofenac, pharmaceutically acceptable diclofenac salts, nimesulide, naproxene, acetaminophen, ibuprofen, ketoprofen and piroxicam; analgesic agents such as salicylates; calcium channel blockers such as nifedipine, amlodipine, and nicardipine; angiotensin converting enzyme inhibitors such as captopril, benazepril hydrochloride, fosinopril sodium, trandolapril, ramipril, lisinopril, enalapril, quinapril hydrochloride, and moexipril hydrochloride; beta-blockers (i.e., beta adrenergic blocking agents) such as sotalol hydrochloride, timolol maleate, esmolol hydrochloride, carteolol, propanolol hydrochloride, betaxolol hydrochloride, penbutolol sulfate, metoprolol tartrate, metoprolol succinate, acebutolol hydrochloride, atenolol, pindolol, and bisoprolol fumarate; centrally active alpha-2-agonists such as clonidine; alpha-1-antagonists such as doxazosin and prazosin; anticholinergic/antispasmodic agents such as dicyclomine hydrochloride, scopolamine hydrobromide, glycopyrrolate, clidinium bromide, flavoxate, and oxybutynin; vasopressin analogues such as vasopressin and desmopressin; antiarrhythmic agents such as quinidine, lidocaine, tocainide hydrochloride, mexiletine hydrochloride, digoxin, verapamil hydrochloride, propafenone hydrochloride, flecainide acetate, procainamide hydrochloride, moricizine hydrochloride, and disopyramide phosphate; antiparkinsonian agents, such as dopamine, L-Dopa/Carbidopa, selegiline, dihydroergocryptine, pergolide, lisuride, apomorphine, and bromocryptine; antiangina agents and antihypertensive agents such as isosorbide mononitrate, isosorbide dinitrate, propranolol, atenolol and verapamil; anticoagulant and antiplatelet agents such as Coumadin, warfarin, acetylsalicylic acid, and ticlopidine; sedatives such as benzodiazepines and barbiturates; ansiolytic agents such as lorazepam, bromazepam, and diazepam; peptidic and biopolymeric agents such as calcitonin, leuprolide and other LHRH agonists, hirudin, cyclosporin, insulin, somatostatin, protirelin, interferon, desmopressin, somatotropin, thymopentin, pidotimod, erythropoietin, interleukins, melatonin, granulocyte/macrophage-CSF, and heparin; antineoplastic agents such as etoposide, etoposide phosphate, cyclophosphamide, methotrexate, 5-fluorouracil, vincristine, doxorubicin, cisplatin, hydroxyurea, leucovorin calcium, tamoxifen, flutamide, asparaginase, altretamine, mitotane, and procarbazine hydrochloride; laxatives such as senna concentrate, casanthranol, bisacodyl, and sodium picosulphate; antidiarrheal agents such as difenoxine hydrochloride, loperamide hydrochloride, furazolidone, diphenoxylate hdyrochloride, and microorganisms; vaccines such as bacterial and viral vaccines; antimicrobial agents such as penicillins, cephalosporins, and macrolides, antifungal agents such as imidazolic and triazolic derivatives; and nucleic acids such as DNA sequences encoding for biological proteins, and antisense oligonucleotides.

Anti-cancer agents include alkylating agents, platinum agents, antimetabolites, topoisomerase inhibitors, antitumor antibiotics, antimitotic agents, aromatase inhibitors, thymidylate synthase inhibitors, DNA antagonists, farnesyltransferase inhibitors, pump inhibitors, histone acetyltransferase inhibitors, metalloproteinase inhibitors, ribonucleoside reductase inhibitors, TNF alpha agonists/antagonists, endothelinA receptor antagonists, retinoic acid receptor agonists, immuno-modulators, hormonal and antihormonal agents, photodynamic agents, and tyrosine kinase inhibitors.

Antibiotics include aminoglycosides (e.g., gentamicin, tobramycin, netilmicin, streptomycin, amikacin, neomycin), bacitracin, corbapenems (e.g., imipenem/cislastatin), cephalosporins, colistin, methenamine, monobactams (e.g., aztreonam), penicillins (e.g., penicillin G, penicillin V, methicillin, natcillin, oxacillin, cloxacillin, dicloxacillin, ampicillin, amoxicillin, carbenicillin, ticarcillin, piperacillin, mezlocillin, azlocillin), polymyxin B, quinolones, and vancomycin; and bacteriostatic agents such as chloramphenicol, clindanyan, macrolides (e.g., erythromycin, azithromycin, clarithromycin), lincomyan, nitrofurantoin, sulfonamides, tetracyclines (e.g., tetracycline, doxycycline, minocycline, demeclocyline), and trimethoprim. Also included are metronidazole, fluoroquinolones, and ritampin.

Enzyme inhibitors are substances which inhibit an enzymatic reaction. Examples of enzyme inhibitors include edrophonium chloride, N-methylphysostigmine, neostigmine bromide, physostigmine sulfate, tacrine, tacrine, 1-hydroxy maleate, iodotubercidin, p-bromotetramiisole, 1O-(alpha-diethylaminopropionyl)-phenothiazine hydrochloride, calmidazolium chloride, hemicholinium-3,3,5-dinitrocatechol, diacylglycerol kinase inhibitor I, diacylglycerol kinase inhibitor II, 3-phenylpropargylamine, N°-monomethyl-Larginine acetate, carbidopa, 3-hydroxybenzylhydrazine, hydralazine, cl orgyline, deprenyl, hydroxylamine, iproniazid phosphate, 6-MeO-tetrahydro-9H-pyrido-indole, nialamide, pargyline, quinacrine, semi carb azide, tranylcypromine, N,N-diethylaminoethyl-2,2-diphenylvalerate hydrochloride, 3-isobutyl-1-methylxanthne, papaverine, indomethacind, 2-cyclooctyl-2-hydroxy ethylamine hydrochloride, 2,3-dichloro-a-methylbenzylamine (DCMB), 8,9-dichloro-2,3,4,5-tetrahydro-1H-2-benzazepine hydrochloride, p-amino glutethimide, p-aminoglutethimide tartrate, 3-iodotyrosine, alpha-methyltyrosine, acetazolamide, dichlorphenamide, 6-hydroxy-2-benzothiazolesulfonamide, and allopurinol.

Antihistamines include pyrilamine, chlorpheniramine, and tetrahydrazoline, among others.

Anti-inflammatory agents include corticosteroids, nonsteroidal anti-inflammatory drugs (e.g., aspirin, phenylbutazone, indomethacin, sulindac, tolmetin, ibuprofen, piroxicam, and fenamates), acetaminophen, phenacetin, gold salts, chloroquine, D-Penicillamine, methotrexate colchicine, allopurinol, probenecid, and sulfinpyrazone.

Muscle relaxants include mephenesin, methocarbomal, cyclobenzaprine hydrochloride, trihexylphenidyl hydrochloride, levodopa/carbidopa, and biperiden.

Anti-spasmodics include atropine, scopolamine, oxyphenonium, and papaverine.

Analgesics include aspirin, phenybutazone, idomethacin, sulindac, tolmetic, ibuprofen, piroxicam, fenamates, acetaminophen, phenacetin, morphine sulfate, codeine sulfate, meperidine, nalorphine, opioids (e.g., codeine sulfate, fentanyl citrate, hydrocodone bitartrate, loperamide, morphine sulfate, noscapine, norcodeine, normorphine, thebaine, nor-binaltorphimine, buprenorphine, chlomaltrexamine, funaltrexamione, nalbuphine, nalorphine, naloxone, naloxonazine, naltrexone, and naltrindole), procaine, lidocain, tetracaine and dibucaine. Ophthalmic agents include sodium fluorescein, rose bengal, methacholine, adrenaline, cocaine, atropine, alpha-chymotrypsin, hyaluronidase, betaxalol, pilocarpine, timolol, timolol salts, and combinations thereof.

Prostaglandins are art recognized and are a class of naturally occurring chemically related long-chain hydroxy fatty acids that have a variety of biological effects.

Anti-depressants are substances capable of preventing or relieving depression.

Examples of anti-depressants include imipramine, amitriptyline, nortriptyline, protriptyline, desipramine, amoxapine, doxepin, maprotiline, tranylcypromine, phenelzine, and isocarboxazide.

Trophic factors are factors whose continued presence improves the viability or longevity of a cell trophic factors include, without limitation, platelet-derived growth factor (PDGP), neutrophil-activating protein, monocyte chemoattractant protein, macrophage-inflammatory protein, platelet factor, platelet basic protein, and melanoma growth stimulating activity; epidermal growth factor, transforming growth factor (alpha), fibroblast growth factor, platelet-derived endothelial cell growth factor, insulin-like growth factor, glial derived growth neurotrophic factor, ciliary neurotrophic factor, nerve growth factor, bone growth/cartilage-inducing factor (alpha and beta), bone morphogenetic proteins, interleukins (e.g., interleukin inhibitors or interleukin receptors, including interleukin 1 through interleukin 10), interferons (e.g., interferon alpha, beta and gamma), hematopoietic factors, including erythropoietin, granulocyte colony stimulating factor, macrophage colony stimulating factor and granulocyte-macrophage colony stimulating factor; tumor necrosis factors, and transforming growth factors (beta), including beta-l, beta-2, beta-3, inhibin, and activin.

Hormones include estrogens (e.g., estradiol, estrone, estriol, diethylstibestrol, quinestrol, chlorotrianisene, ethinyl estradiol, mestranol), anti-estrogens (e.g., clomiphene, tamoxifen), progestins (e.g., medroxyprogesterone, norethindrone, hydroxyprogesterone, norgestrel), antiprogestin (mifepristone), androgens (e. g, testosterone cypionate, fluoxymesterone, danazol, testolactone), anti-androgens (e.g., cyproterone acetate, flutamide), thyroid hormones (e.g., triiodothyronne, thyroxine, propylthiouracil, methimazole, and iodixode), and pituitary hormones (e.g., corticotropin, sumutotropin, oxytocin, and vasopressin). Hormones are commonly employed in hormone replacement therapy and/or for purposes of birth control. Steroid hormones, such as prednisone, are also used as immunosuppressants and anti-inflammatoires. In some aspects, the additive is an agent that stimulates tissue formation, and/or healing and regrowth of natural tissues, and any combinations thereof. Agents that increase formation of new tissues and/or stimulates healing or regrowth of native tissue at the site of injection can include, but are not limited to, fibroblast growth factor (FGF), transforming growth factor-beta (TGF-beta, platelet-derived growth factor (PDGF), epidermal growth factors (EGFs), connective tissue activated peptides (CTAPs), osteogenic factors including bone morphogenic proteins, heparin, angiotensin II (A-II) and fragments thereof, insulin-like growth factors, tumor necrosis factors, interleukins, colony stimulating factors, erythropoietin, nerve growth factors, interferons, biologically active analogs, fragments, and derivatives of such growth factors, and any combinations thereof.

In some aspects, the silk composition can further comprise at least one additional material for soft tissue augmentation, e.g., dermal filler materials, including, but not limited to, poly(methyl methacrylate) microspheres, hydroxylapatite, poly(L-lactic acid), collagen, elastin, and glycosaminoglycans, hyaluronic acid, commercial dermal filler products such as BOTOX® (from Allergan), DYSPORT®, COSMODERM®, EVOLENCE®, RADIESSE®, RESTYLANE®, JUVEDERM® (from Allergan), SCULPTRA®, PERLANE®, and CAPTIQEIER, and any combinations thereof.

In some aspects, the additive is a wound healing agent. As used herein, a “wound healing agent” is a compound or composition that actively promotes wound healing process.

Exemplary wound healing agents include, but are not limited to dexpanthenol; growth factors; enzymes, hormones; povidon-iodide; fatty acids; anti-inflammatory agents; antibiotics; antimicrobials; antiseptics; cytokines; thrombin; angalgesics; opioids; aminoxyls; furoxans; nitrosothiols; nitrates and anthocyanins; nucleosides, such as adenosine; and nucleotides, such as adenosine diphosphate (ADP) and adenosine triphosphate (ATP); neutotransmitter/neuromodulators, such as acetylcholine and 5-hydroxytryptamine (serotonin/5-HT); histamine and catecholamines, such as adrenalin and noradrenalin; lipid molecules, such as 5 sphingosine-1-phosphate and lysophosphatidic acid; amino acids, such as arginine and lysine; peptides such as the bradykinins, substance P and calcium gene-related peptide (CGRP); nitric oxide; and any combinations thereof.

In certain aspects, the active agents provided herein are immunogens. In one aspect, the immunogen is a vaccine. Most vaccines are sensitive to environmental conditions under which they are stored and/or transported. For example, freezing may increase reactogenicity (e.g., capability of causing an immunological reaction) and/or loss of potency for some vaccines (e.g., HepB, and DTaP/IPV/FQB), or cause hairline cracks in the container, leading to contamination. Further, some vaccines (e.g., BCG, Varicella, and MMR) are sensitive to heat. Many vaccines (e.g., BCG, MMR, Varicella, Meningococcal C Conjugate, and most DTaP-containing vaccines) are light sensitive. See, e.g., Galazka et ak, Thermostability of vaccines, in Global Programme for Vaccines & Immunization (World Health Organization, Geneva, 1998); Peetermans et ak, Stability of freeze-dried rubella virus vaccine (Cendehill strain) at various temperatures, 1 J. Biological Standardization 179 (1973). Thus, the compositions and methods provided herein also provide for stabilization of vaccines regardless of the cold chain and/or other environmental conditions.

In some aspects, the additive is a cell, e.g., a biological cell. Cells useful for incorporation into the composition can come from any source, e.g., mammalian, insect, plant, etc. In some aspects, the cell can be a human, rat or mouse cell. In general, cells to be used with the compositions provided herein can be any types of cells. In general, the cells should be viable when encapsulated within compositions. In some aspects, cells that can be used with the composition include, but are not limited to, mammalian cells (e.g. human cells, primate cells, mammalian cells, rodent cells, etc.), avian cells, fish cells, insect cells, plant cells, fungal cells, bacterial cells, and hybrid cells. In some aspects, exemplary cells that can be used with the compositions include platelets, activated platelets, stem cells, totipotent cells, pluripotent cells, and/or embryonic stem cells. In some aspects, exemplary cells that can be encapsulated within compositions include, but are not limited to, primary cells and/or cell lines from any tissue. For example, cardiomyocytes, myocytes, hepatocytes, keratinocytes, melanocytes, neurons, astrocytes, embryonic stem cells, adult stem cells, hematopoietic stem cells, hematopoietic cells (e.g. monocytes, neutrophils, macrophages, etc.), ameloblasts, fibroblasts, chondrocytes, osteoblasts, osteoclasts, neurons, sperm cells, egg cells, liver cells, epithelial cells from lung, epithelial cells from gut, epithelial cells from intestine, liver, epithelial cells from skin, etc, and/or hybrids thereof, can be included in the silk/platelet compositions disclosed herein. Those skilled in the art will recognize that the cells listed herein represent an exemplary, not comprehensive, list of cells. Cells can be obtained from donors (allogenic) or from recipients (autologous). Cells can be obtained, as a non-limiting example, by biopsy or other surgical means known to those skilled in the art.

In some aspects, the cell can be a genetically modified cell. A cell can be genetically modified to express and secrete a desired compound, e.g. a bioactive agent, a growth factor, differentiation factor, cytokines, and the like. Methods of genetically modifying cells for expressing and secreting compounds of interest are known in the art and easily adaptable by one of skill in the art.

Differentiated cells that have been reprogrammed into stem cells can also be used.

For example, human skin cells reprogrammed into embryonic stem cells by the transduction of Oct3/4, Sox2, c-Myc and Klf4 (Junying Yu, et. ah, Science, 2007, 318, 1917-1920 and Takahashi K. et. ah, Cell, 2007, 131, 1-12).

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

While the invention has been illustrated and described in detail in the foregoing drawings and description, the same is to be considered as illustrative and not restrictive in character, it being understood that only illustrative embodiments thereof have been shown and described and that all changes and modifications that come within the spirit of the invention are desired to be protected.

For example, any of the features or functions of any of the embodiments disclosed herein may be incorporated into any of the other embodiments disclosed herein.

### EXAMPLES

Material versatility, chemistry and property modulation, composites: Multiple synthetic mucin analogs based on silk proteins are prepared leveraging different established chemistries, where different MUCIN2 (MUC2: mucin present in gut) relevant sugars are covalently appended on different locations of silk protein to yield different versions with different degrees of sugar substitution, and mechanical property can be tunable when supplemented with silk protein in composite setting.

Selective impact on barrier function: Among different glycopolymers variants, GalNAc and GalN modified silk glycopolymers, singularly, retain the barrier integrity for up to seven days. Among composite formulations, both versions of GalNAc modified silk (SF(S)-GalNAc, SF (D, E)-GalNAc) in composition with silk retain monolayer integrity. In addition, GlcN and GalN modified silk in composition with silk also display protective function up to seven days. NeuNAc and GlcNAc modified silk (both alone and in composite) do not display barrier protection.

Selective impact on gut pathogens and commensals: GalNAc modified silk (SF(S)-GalNAc, when supplemented with silk (0.8% SF(S)-GalNAc+1.2 % SF) provide adequate barrier to pathogenic strains of E. coli (Adherent invasive E. coli) while allowing commensal bacteria (E. coli Nissle) to reside within the mucin mimetic layer, showing selective barrier integrity for pathogens and commensal. Studies on impact of other analogs on gut pathogens and commensal are disclosed.

Organoid culture: The study utilized human intestinal organoids, which have gained considerable recognition in research due to their ability to be derived directly from patients and closely replicate the physiological characteristics of the human gut, which has not yet explored for study of mucin analogs. These organoids serve as valuable models for studying various aspects of intestinal function. However, their application in investigating the potential for supporting and interacting with artificial mucins, which play a crucial role in the formation of protective barriers in the gut, has been relatively unexplored. By employing human intestinal organoids, this study can bridge this gap and examine the feasibility of utilizing organoids as a platform for studying the dynamic interaction between patient-derived intestinal epithelium and artificial mucins.

Scaffold: The study incorporated a 3D scaffold system, which played a pivotal role in enhancing the versatility (half scaffold and full tubular scaffold designs, static and dynamic cultures) and applicability of the experimental setup. This scaffold system allowed for the incorporation of various cell types, expanding beyond the limitations of the specific cell types mentioned, such as epithelial cells, myofibroblasts, immune cells and neuron cells. The flexibility of this system enabled the integration of diverse cell populations, facilitating investigations into complex cellular interactions within a three-dimensional environment. This capability opens new avenues for studying the interplay between different cell types and their responses to external stimuli, such as mucin mimetics and microbiome, fostering a deeper understanding of the intricacies of tissue development and function.

### Materials Preparation

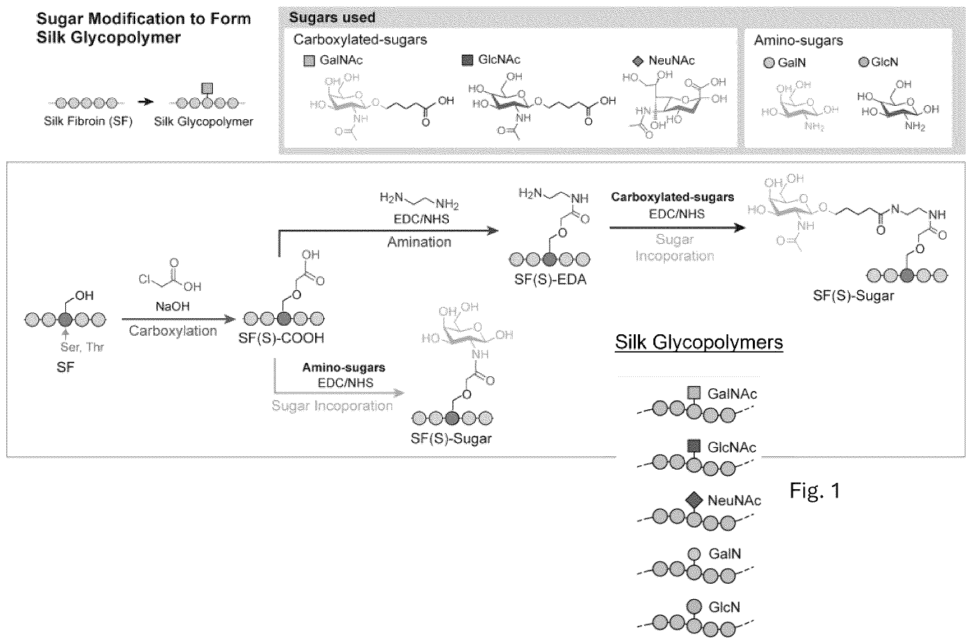

Different silk glycopolymers variants (FIG. 1) were prepared by using different chemistries (carboxylation, carbodiimide coupling). Briefly, carboxylated versions of acetylated sugars (GalNAc, GlcNAc, NeuNAc) are carbodiimide coupled with the aminated silk (SF(S)-EDA) to get the corresponding silk glycopolymers. Similarly, amino sugars like galactosamine (GalN) and glucosamine (GlcN) are covalently incorporated by carbodiimide coupling them with the carboxylated sugar (SF(S)-COOH) to synthesize the corresponding silk glycopolymers. Since serine constitutes 12.3 mol % of total amino acids present on silk, different sugars are incorporated onto the serine residues of the silk protein to maximize the sugar content in the resulting glycopolymers.

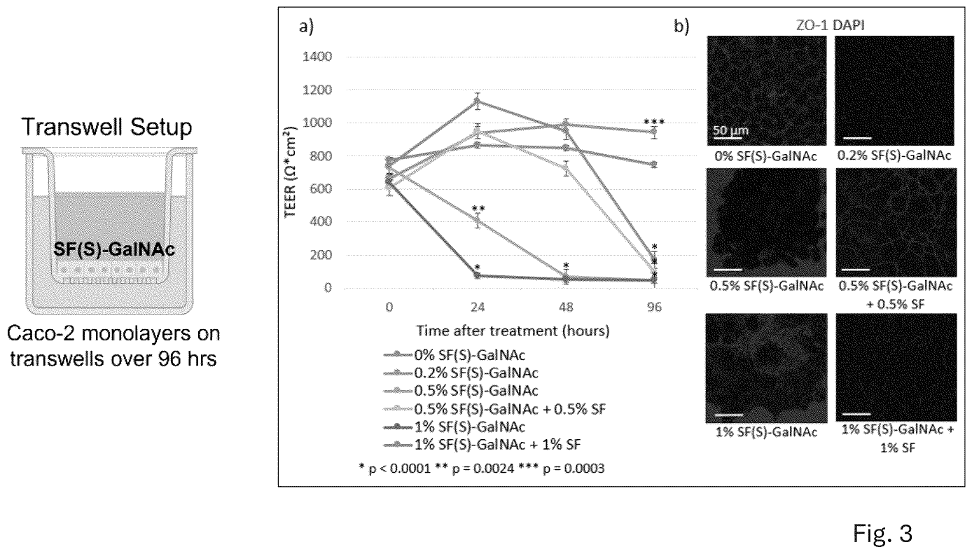

GalNAc modified silk polymer (SF(S)-GalNAc) was initially tested at different concentrations to investigate the monolayer integrity by trans-epithelial electrical resistance (TEER) in a transwell set up (FIG. 3). Caco-2 cells were used for the study. A maximum concentration of 2 wt % of silk glycopolymers were chosen like the native mucin concentration in gut mucus. As such, five different formulations with different concentrations (three glycopolymers only: 0.2, 0.5, 1% SF(S)-GalNAc) and two in composite formulations with native silk fibroin (SF) solution (0.5:0.5. 1:1 (SF(S)-GalNAc: SF)). Among all the concentrations, different composite formulations and control, SF only and 0.2% SF(S)-GalNAc maintain the monolayer integrity of Caco2 cell until 96 h according to TEER data (FIG. 3a, b). Also, to note, in addition to SF only and 0.2% SF(S)-GalNAc, composite formulations (0.5:0.5 and 1:1 SF and SF(S)-GalNAc) also maintain the monolayer integrity until 48 h.

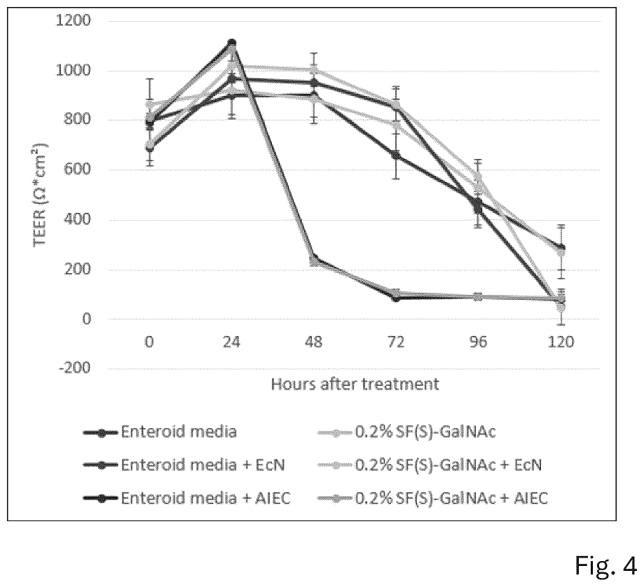

Based on the above preliminary TEER resistance findings, 0.2% SF(S)-GalNAc was screened for further bacterial studies. Briefly, enteroid monolayers are cultured with commensal E. coli Nissle (EcN) and pathogenic adherent-invasive E. coli (AIEC) in a transwell system (FIG. 4). 0.2% of SF(S)-GalNAc was exposed to the culture in solution format for five days. TEER data demonstrated no resistance of the glycopolymers to the pathogenic AIEC as it does not provide any adequate protective layers after 24 h. However, the glycopolymers provides monolayer integrity against commensal EcN for at least 48 h. (FIG. 4) From the above results, it can be concluded that, to better mimic the in vivo protective property of gut mucus, the total concentration of the mucin-analogs should be increased to provide a thicker layer and match the concentration of mucin (MUC2) in gut mucus (2 wt %).

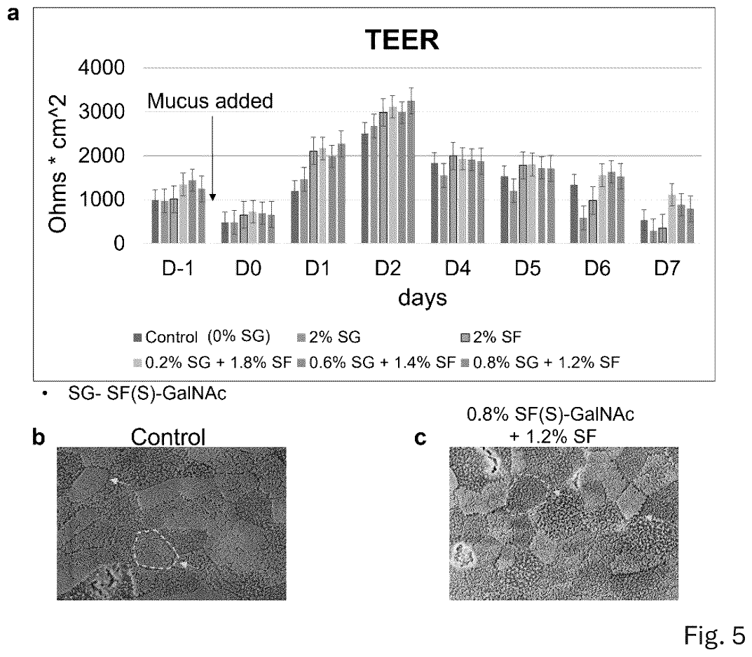

Based on the result and analysis, we increased the total polymer concentration to 2 wt % to match the mucin concentration in natural gut mucus. As such, we tested the TEER resistance of 2% only SF(S)-GalNAc and SF only and three different ratios of SF and SF(S)-GalNAc in composite settings in addition to control. The ratios of SF(S)-GalNAc and SF include 0.2:1.8, 0.6:1.4 and 0.8:1.2 wt % respectively (FIG. 5a). We did not observe any significant effect on TEER for the glycopolymers, composites and SF for only up to five days. The composite formulations can maintain the monolayer integrity for up to seven days as assessed by their TEER data (FIG. 5a). Scanning electron microscopy (SEM) images (FIG. 5b, c) validated the TEER findings where we observed the intact monolayers with microvilli. We have observed microvilli in the control and in the 0.8:1.2 SF(S)-GalNAc: SF composite formulation.

Assessment of protective function and epithelial monolayer integrity was further expanded to a library of different sugar functionalized silk glycopolymers to analyze their TEER performance and further screening for interaction with different gut microbes. We had incorporated five different mucin2 (MUC2) relevant sugars onto silk protein and the sugars are placed on serine, aspartic and glutamic acid units using a combination of different substitution and bioconjugation chemistries.

While the advantage of sugar placement on serine moieties includes possibilities of higher substitution rates (due to availability of more serine units: 12.1 mol % or 635 residues in heavy chain silk fibroin), the limitations include chain scission due to harsh reaction condition (higher pH) that result in decrease in molecular weight which affects their mechanical properties. To overcome this, we have covalently appended GalNAc directly onto pre-synthesized aminated silk fibroin (SF (D, E)-EDA). EDA was modified onto SF in the presence of EDC and NHS in a similar manner as described above, by leveraging the carboxylic acid residues present in silk chain due to aspartic and glutamic acid (1.1 mol %, 55 units). No decrease in molecular weight is expected for the glycopolymer prepared in this method (SF (D, E)-GalNAc) as the modification steps did not involve high pH. However, low degree of sugar substitution is expected in this method because of unavailability of enough active sites (i.e., primary amines) for carbodiimide coupling in comparison to SF(S)-EDA, which is aminated after serine carboxylation. As such, SF (D, E)-GalNAc is expected to impart better mechanical properties and thickness (thereby more protection) in comparison to glycopolymers where sugars are covalently grafted leveraging serine carboxylation under similar polymer concentration. Adding this glycopolymer is important to the pool of serine substituted glycopolymers for their protective function assessment as it facilitates deciphering the role of mechanical properties and degree of sugar substitution to the native function.

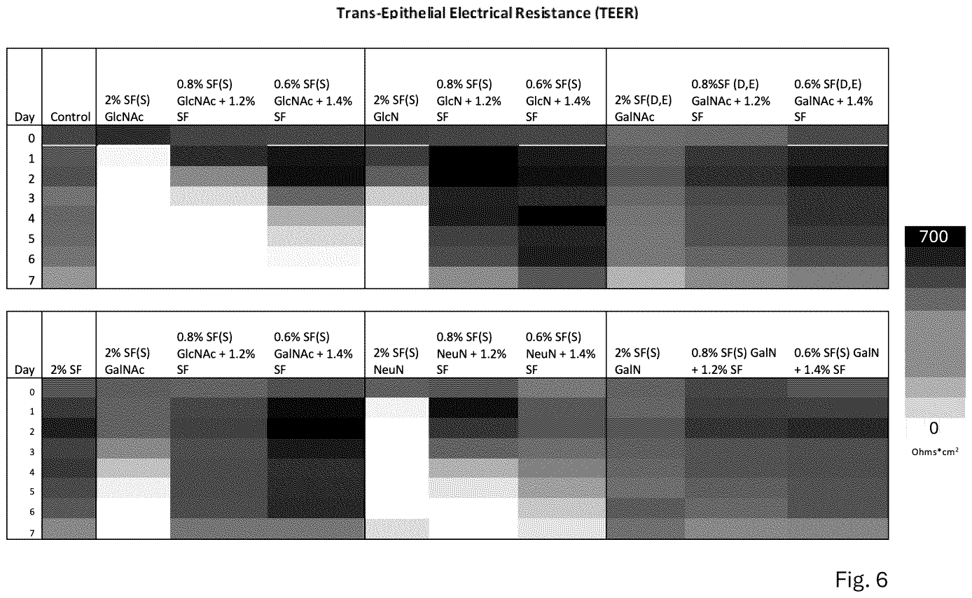

From the above TEER experiment (FIG. 5), we had screened three formulations (2% SF(S)-GalNAc, 0.6:1.4, 0.8:1.2 SF(S)-GAINAc SF) that retain the monolayer integrity of the Caco2 epithelial layer up to seven days. These polymer formulations were applied to the pool of six glycopolymers and in total 18 different glycopolymers (alone and in composition with SF) and only SF (2%) as control, were assessed for their ability to retain monolayer integrity by TEER studies (FIG. 6).

Among all the silk glycopolymers variants, SF (D, E)-GalNAc and SF(S)-GalN did not impact any significant effect on the TEER up to seven days both singularly and when supplemented with silk solution. All formulations of GlcNAc and NeuNAc modified glycopolymers did not retain the monolayer integrity. Similarly, SF(S)-GalNAc and SF(S)-GlcN when exposed alone did not retain the monolayer integrity after 2 days; however, supplementing them with SF solutions significantly improved their TEER performance. This could be related to their mechanical properties, as SF increased the mechanical properties and thickness of serine modified glycopolymers. The role of mechanical properties on the protective function of these analogs could be further elucidated by comparing the TEER performance of two different GalNAc substituted silk polymer, albeit on different amino acids: SF(S)-GalNAc and SF (D, E)-GalNAc. When treated alone, SF(S)-GalNAc did not protect the monolayer integrity while SF (D, E)-GalNAc protected the epithelial layer for up to seven days. As described above, SF (D, E)-GalNAc was expected to possess better mechanical properties in the same condition. TEER results also demonstrated the effect of different variants like glycopolymers concentration, mechanical properties, and type of sugars present in silk chain on the overall protective function.

1. A cultured composition comprising:
an integral layer of intestinal epithelial cells;
an artificial mucus atop the integral layer of intestinal epithelial cells, wherein the artificial mucus comprises a silk glycopolymer and optionally a silk polymer lacking sugar substituents, the artificial mucus having a degree of substitution of the silk glycopolymer, a location of substitution of the silk glycopolymer, a concentration of the silk glycopolymer, an overall polymer concentration, an overall silk polymer concentration, an optional concentration of the silk polymer lacking sugar substituents, an optional ratio by weight of the silk glycopolymer and the silk polymer lacking sugar substituents, and/or a silk glycopolymer silk backbone molecular weight is tailored to maintain the integral layer of intestinal epithelial cells for a predetermined length of time; and
growth media accessible to the intestinal epithelial cells and tailored to maintain viability of the intestinal epithelial cells.

2. The cultured composition of claim 1, the cultured composition further comprising a three-dimensional tissue scaffold, wherein the integral layer of intestinal epithelial cells is affixed to the three-dimensional tissue scaffold.

3.-6. (canceled)

7. The cultured composition of claim 2, wherein the three-dimensional tissue scaffold is a silk fibroin tissue scaffold.

8. The cultured composition of claim 1, the cultured composition further comprising a plurality of intestinal organoids physiologically associated with the integral layer of intestinal epithelial cells.

9. The cultured composition of claim 8, wherein the plurality of intestinal organoids is patient-specific.

10. The cultured composition of claim 9, wherein the intestinal organoids of the plurality of intestinal organoids are harvested from a specific patient or grown from cells from the specific patient.

11. The cultured composition of claim 10, wherein the specific patient has a predefined disease state.

12. The cultured composition of claim 11, wherein the predefined disease state is inflammatory bowel disease (IBD).

13. The cultured composition of claim 1, wherein the integral layer of intestinal epithelial cells is an integral layer of Caco-2 cells.

14. The cultured composition of claim 1, wherein the silk glycopolymer has a degree of sugar substitution of between 0.5% and 40%.

15.-17. (canceled)

18. The cultured composition of claim 1, wherein the silk glycopolymer is an O-glycan-substituted silk fibroin.

19. The cultured composition of claim 1, wherein the silk glycopolymer comprises a GalNAc-modified silk fibroin, a GlcN-modified silk fibroin, and/or a GalN-modified silk fibroin.

20.-21. (canceled)

22. The cultured composition of claim 1, wherein the silk glycopolymer comprises SF(D, E)-GalNAc, SF(S)-GalN, and/or SF(S)-GlcN.

23.-24. (canceled)

25. The cultured composition of claim 1, wherein artificial mucus has a total polymer concentration in a weight percentage of between 0.5% and 10.0%, an overall silk polymer concentration in a weight percentage of between 0.5% and 10.0%, and/or a concentration of the silk glycopolymer in a weight percentage of between 0.5% and 10.0%.

26.-27. (canceled)

28. The cultured composition of claim 1, wherein the artificial mucus comprises the silk polymer lacking sugar substituents.

29. The cultured composition of claim 1, wherein the artificial mucus has a concentration of the silk polymer lacking sugar substituents in a weight percentage of between 0.1% and 4.0%, or wherein a ratio by weight of the silk glycopolymer to the silk polymer lacking sugar substituents is between 1:5 and 5:1.

30.-33. (canceled)

34. The cultured composition of claim 1, wherein the artificial mucus has one or more mechanical properties within 5% of a respective native mucus mechanical property.

35. The cultured composition of claim 1, wherein the integral layer of intestinal epithelial cells maintains integrity for at least 7 days.

36. The cultured composition of claim 1, wherein the artificial mucus is tailored to maintain integrity of the integral layer of intestinal epithelial cells in a microbial environment that is otherwise destructive to the integral layer of intestinal epithelial cells.

37. A method of using the cultured composition of claim 1, the method comprising: observing integrity of the integral layer of intestinal epithelial cells.

38.-40. (canceled)
